(19) United States
(12) Patent Application Publication
(10) Pub. No.: US 2020/0385693 A1
(43) Pub. Date: Dec. 10, 2020

(54) METHODS OF PRODUCING ENZYMES USING PICHIA CELLS

(71) Applicant: King Abdullah University of Science and Technology, Thuwal (SA)

(72) Inventors: Jasmeen S. Merzaban, Thuwal (SA); Kosuke Sakashita, Thuwal (SA); Asma Saeed Al-Amoodi, Thuwal (SA); Amal Ali, Thuwal (SA); Muhammad Tehseen, Thuwal (SA)

(21) Appl. No.: 16/769,979

(22) PCT Filed: Dec. 3, 2018
(86) PCT No.: PCT/IB2018/059574

§ 371 Date: Jun. 4, 2020

Related U.S. Application Data
(60) Provisional application No. 62/594,362, filed on Dec. 4, 2017.
(60) Provisional application No. 62/608,935, filed on Dec. 21, 2017.
(60) Provisional application No. 62/772,186, filed on Nov. 28, 2018.

(51) Int. Cl. C12N 9/10; C12N 15/81

(52) U.S. Cl. CPC C12N 9/1051; C12Y 204/01214; C12N 15/815

(57) ABSTRACT

Provided are methods for recombinantly producing enzymatically active glycosyltransferase (GT) enzymes. Active recombinant glycosyltransferase enzymes and method of use thereof are also provided. The methods for recombinantly producing enzymatically active GTs relies on a yeast expression system, preferably, a Pichia pastoris, expression system and more preferably, a Pichia pastoris stain with an ade2 deletion. Recombinantly produced enzymatically active GT enzymes produced according to the methods disclosed herein can be used for cell surface glycan engineering. The method includes contacting a cell with the disclosed compositions comprising purified recombinant GT enzyme and a substrate (nucleotide sugar) for the GT enzyme for an effective time for the GT enzyme to catalyze transfer of its substrate onto an acceptor site at the surface of the cell. The composition in preferred embodiments does not include glycerol as a stabilizer or it includes at least 50% glycerol.

### CROSS-REFERENCED TO RELATED APPLICATIONS

This application claims the benefit of and priority to U.S. Provisional Application No. 62/594,362 filed Dec. 4, 2017, 62/608,935 filed Dec. 21, 2017, and 62/772,186 filed Nov. 28, 2018, which are hereby incorporated herein by reference in their entirety.

### FIELD OF THE INVENTION

### Field of the Invention

The invention relates generally to recombinant expression systems and more specifically to methods of producing recombinant glycosyltransferases in a Pichia pastoris expression system, the recombinantly produced enzymes and uses thereof.

### Background of the Invention

Cell migration is an important process involved in a variety of physiological and pathological functions such as attracting immune cells to inflammatory sites, migration and engraftment of therapeutic stem cells to their target tissue, and metastasis of cancer cells. The mechanism of delivery of cells to these sites in the context of inflammation and/or injury is a sophisticated process that is controlled by a number of adhesion molecules including the selectins, chemokines and integrins which all function in a coordinated stepwise manner.

Cell migration begins with tethering and rolling of flowing cells onto the endothelial cells within the vasculature which is mainly mediated by the selectins and their ligands. It causes the tethered cell to roll along the endothelium at a slower speed. Next, chemokine binding to its receptor on the flowing cells leads to integrin activation on the cell in flow. This activation leads to conformational changes in the integrin by inside-out signal transduction events resulting in high-affinity binding of the integrin to their cellular adhesion molecules (CAMs) on the endothelium. This results in firm adhesion and arrest of the cell that was in flow onto the endothelial cells. Ultimately the last step follows where the cell transmigrates to reach the extravascular space. Although each step in this process is important and dependent on the previous step, the interaction of selectins with their ligands are the gatekeepers of the multistep paradigm. Selectins are type-I transmembrane C-type (Ca²⁺-dependent) lectins that bind to carbohydrate ligands in a calcium-dependent manner. The binding of selectins with their ligands mainly depends on the lectin domain. All selectins have affinity towards Sialyl Lewis (sLe^{x}) carbohydrate structures. The fucose and sialic acid of this 4-sugar structure provide the negative charge for binding to positively charged amino acids that are found in all selectins. In addition to the sLe^{x}, P- and L-selectin also require sulfation of nearby tyrosines or sugars respectively. Selectins bind to specified terminal carbohydrate determinants that are composed of tetrasaccharide sialyl Lewis x (sLe^{x}; or also to its isomer sLe^{a}). sLe^{x} is a sialofucosylated sugar comprised of a sialic acid linked to galactose in an α(2,3) bond and a fucose linked to a N-acetylglucosamine in an α(1,3) bond. Both fucosylation and sialylation are essential for binding to selectins. These determinants could be displayed on either a protein scaffold (i.e., a glycoprotein) or a lipid scaffold (i.e., a glycolipid).

Cells used for cell therapy, for example, stem cells, usually lack expression of GT enzymes, the expression of which could improve migration following implantation. Purified GTs may be used to create sLe^{x} structures on therapeutic cells such as mesenchymal stem cells and HSPCs to promote their migration to target organs. However, prior methods of recombinantly producing GT present with various limitations ranging from inability to produce active enzyme, to difficulty in obtaining enzyme with high yield, activity, or processes that are not cost effective for large scale enzyme production. For example, although relatively practical and simple with large potential yields, bacterial expression systems do not result in enzymatically active GTs likely due to the absence of glycosylation machinery required for enzymatic activity i.e. N-glycosylation.

Accordingly, there is still a need for methods to recombinantly produce active glycosyltransferase enzymes.

It is therefore an object of the present invention to provide methods for recombinantly producing enzymatically active glycosyltransferase enzymes.

It is also an object of the present invention to provide enzymatically active GT.

It is a further object of the present invention to provide a method for cell surface glycan engineering.

It is still an object of the present invention to provide a method for improving migration of implanted cells.

### SUMMARY OF THE INVENTION

Provided are methods for recombinantly producing enzymatically active glycosyltransferase (GT) enzymes and expression systems for recombinantly producing GTs. Also provided are active recombinant glycosyltransferase enzymes and method of use thereof.

The methods for recombinantly producing enzymatically active GTs relies on a yeast expression system, preferably, a Pichia pastoris, expression system and more preferably, an expression system that uses a Pichia pastoris stain with an ade2 deletion. This strain is an ADE2 auxotroph that is unable to grow in the absence of adenine because of full deletion of the ADE2 gene and part of its promoter. The ADE2 gene encodes phosphoribosylaminoimidazole carboxylase, which catalyzes the sixth step in the de novo biosynthesis of purine nucleotides. The method includes genetically engineering a host organism to express a GT enzyme, preferably, the luminal domain of the GT enzyme, comprising its catalytic domain. The host organism is Pichia pastoris more preferably, a Pichia pastoris stain with an ade2 deletion. The method includes introducing into the Pichia pastoris host, a vector containing gene encoding the catalytic domain of the GT enzyme, operably linked to one or more expression control sequences and a Pichia pastoris secretion signal, preferably, the α-mating factor. The vector preferably comprises a 6×Histidine (His)-tag added to the N-terminus of the gene encoding the GT enzyme. A particularly preferred GT transferase enzyme is human alpha-(1,3)-fucosyltransferase, more preferably, human alpha-(1,3)-fucosyltransferase 6 (FUT6). The method further comprises purifying the recombinantly expressed GT enzyme from the host cells, using immobilized metal affinity chromatography (IMAC) as a preferred purification method.

Recombinant Pichia pastoris for producing active glycosyltransferase enzymes are provided. The Pichia pastoris more preferably, a Pichia pastoris strain with an ade2 deletion comprising a vector containing a gene encoding the catalytic domain of a GT enzyme, operably linked to one or more expression control sequences and to a Pichia pastoris secretion signal, preferably, the α-mating factor. The vector preferably comprises a 6×Histidine (His)-tag added to the N-terminus of the gene encoding the GT enzyme. A particularly preferred gene encoding a GT transferase enzyme is gene encoding human alpha-(1,3)-fucosyltransferase, more preferably, amino acids 35-359 of human alpha-(1,3)-fucosyltransferase 6 (FUT6).

It is also an object of the present invention to provide a composition comprising recombinantly produced enzymatically active GT enzyme. The composition comprises purified recombinant GT enzyme in an acceptable buffer, comprising at least 50% glycerol as a stabilizer. Preferably the composition comprises 0% glycerol as a stabilizer and a cation, for example manganese. In a particularly preferred embodiment, the enzyme composition is lyophilized.

It is a further object of the present invention to provide a method for cell surface glycan engineering. The method includes contacting a cell with the disclosed compositions comprising purified recombinant GT enzyme and a substrate (nucleotide sugar) for the GT enzyme for an effective time for the GT enzyme to catalyze transfer of substrate onto an acceptor site at the surface of the cell. The composition in preferred embodiments does not include glycerol as a stabilizer or it includes at least 50% glycerol. In a particularly preferred embodiment, the enzyme compositions include 0% glycerol and Mn added in concentrations between 3-4 mM.

It is still an object of the present invention to provide a method for improving migration of implanted cells. The method includes contacting a cell in need thereof, with a composition comprising a recombinant GT enzyme and a substrate for the GT enzyme for an effective amount of time to catalyze transfer of the substrate onto an acceptor site on the cell.

### BRIEF DESCRIPTION OF THE DRAWINGS

FIG. 1A is a schematic showing the different domains of the selection family of adhesion molecules. The selectin family of adhesion molecules share a common structure composed of five different domains: lectin binding domain, (epidermal growth factor) EGF domain, consensus repeat, transmembrane region and short cytoplasmic tail.

FIG. 1B is a schematic of FUTs structure with a short NH2 terminal tail in the cytosol followed by a transmembrane domain and stem region which is linked to the catalytic domain in the Golgi lumen. FIG. 1C is a schematic showing GTs involved in the formation of sLe^{a} and sLe^{x} structures on selectin ligands. The expression of the GTs responsible for capping the galactose (Gal) of the type 1 or type 2 lactosamines [Galβ1,4GlcNAc or Galβ1,3GlcNAc] with sialic acid (NeuAc)—sialyltransferases (ST)—and the terminal N-acetylglucosamine (GlcNAc) of the lactosamine with fucose (Fuc)—fucosyltransferases (FUT)—to create sLe^{x} are often correlated with cells that migrate or home to tissues where selectins are expressed.

FIG. 2 shows the pPink-aHC Plasmid map and integration of human FUT6 sequence to the plasmid with specific digestion with assigned restriction enzymes, GOI: is human FUT6.

FIGS. 3A and 3B show purification of FUT6 expressed in P. pastoris. Neat (FIG. 3A) and 60-fold concentrated (FIG. 3B) samples relating to the purification of histidine tagged FUT6 enzyme were run on a 4-20% polyacrylamide gel and stained with Coomassie blue. 1, protein ladder; 2, crude extract; 3, Flow through; 4, 5 mM imidazole washing fraction; 5, 250 mM imidazole elution fraction; 6, 400 mM imidazole elution fraction. The arrows refer to the potential molecular weight of the FUT6 enzyme. FIG. 3C shows western-blot analysis of purified FUT6 protein from P. pastoris cultures. The concentrated eluate following purification of P. pastoris cells was run on an SDS-PAGE gel and transferred to a PVDF membrane prior to blotting with either anti-FUT6 antibody (1:1000; Abcam). To detect the primary antibodies, incubation with goat anti-rabbit horseradish peroxidase (1:20000) secondary antibody was used. FIG. 3D is a western blot showing the determination of FUT6 concentration using BSA standards. A range of known concentrations of BSA were used to determine the concentration of FUT6 in the purified eluate. The SDS-PAGE gel was stained with Coomassie in order to highlight the protein bands. Lane 1: protein ladder; Lane 2: 2 mg/mL BSA; Lane 3: 1.5 mg/mL; Lane 4: 1.0 mg/mL; lane 5: 0.750 mg/mL; Lane 6: 0.500 mg/mL; Lane 7: 0.250 mg/mL; Lane 8: 0.125 mg/mL; Lane 9: 0.025 mg/mLBSA. Lane 10 corresponds to 10 μL of the purified recombinant FUT6.

FIG. 4A is a general scheme of the principle used to determine the FUT6 activity. FIG. 4B is a line graph showing the GDP standard curve prepared at the indicated GDP concentration range in 25 μl of GT reaction buffer FIGS. 4C and 4D show biochemical characterization of FUT6 using bioluminescent GDP Glo assay. FIG. 4C: the amount of GDP product in pmol with luminescence signal; FIG. 4D: FUT6 titrated in six serial dilutions with luminescence signal. FIG. 4E shows specific activity of FUT6. Specific activity was calculated using the amount of GDP produced from a standard curve (FIG. 4B) that was prepared on the same plate with a titrated amount of FUT6 enzyme.

FIG. 5A shows Flow cytometric analysis of sLe^{x} expression. K562 cells were treated with the appropriate concentration of purified FUT6 in HBSS, 0.1% human serum albumin, 0.5 mM GDP-Fucose, 5 mM MnCl2 and 25 mM HEPES pH 7.5 and incubated for 30 min at 37° C. Further cells were washed and stained with HECA452 antibody prior to analysis using the BD FACS Canto II. FIG. 5B shows PSGL-1, CD43 and CD44 expression in K562 cells. K562 cells were stained for antibodies specific to PSGL-1, CD43, CD34 and CD44. Black, isotype control (mouse IgG and mouse IgG2a); Light Gray, antibodies specific for each ligand. FIG. 5C shows E-selectin ligands created following FUT6 treatment of K562 cells. K562 cells lysate that were either untreated (−) or treated (+) with FUT6 to express sLe^{x} were prepared for Western blot analysis and blotted E-Ig (left panel) or with HECA452 (right panel) to determine E-selectin binding and sLe^{x} expression respectively. Lane 1,2, ladders; 3, FUT6 treated K562 cells; 4, untreated K562 cells; 5, Kgla cells lysate. FIG. 5D shows western blot of CD44 and CD43 immune-purified before and after treatment with FUT6-K562. CD44 and CD43 were immuno-purified from FUT6-K562 cells and Untreated K562 cells. The immuno-purified proteins were then prepared for Western blot and stained with either HECA452 or E-Ig as well as for each immuno-purified protein.

FIG. 6A shows flow cytometric analysis of human MSCs markers, CD105 (clone 43A3) and CD73 (clone AD2), are shown (black line). Mouse IgG isotype control is shown as a gray line. FIG. 6B is a western blot analysis for HECA-452 or E-Ig. MSCs were either treated (+) with purified rhFTVI (HBSS, 0.1% human serum albumin, 0.5-mM GDP-Fucose, 5-mM MnCl2 and 25-mM HEPES pH=7.5; treated) or in buffer alone (−) and incubated for 30-min at 37° C. The cells were then lysed and prepared for Western blot analysis for HECA-452 or E-Ig. FIG. 6C shows flow cytometric analysis for sLe^{x} expression and E-Ig binding. Following MSCs treatment with rhFTVI, flow cytometric analysis for sLe^{x} expression (HECA-452, CD15s) and E-Ig binding was determined. Red: untreated MSCs incubated with buffer only without rhFTVI; Blue: MSCs treated with rhFTVI in the presence of 2-mM Ca²⁺; Green: MSCs treated with rhFTVI in the presence of 10-mM EDTA.

FIG. 7A shows Human iPS cells (left panel) (differentiated toward HSPCs showing 30% CD34⁺ cells were generated following differentiation of iPS cells to HSPCs) double stained for CD34 surface antigen and HECA-452 antigenic determinant. The lack of sLe^{x/a} structures and E-selectin binding on iPS-HSPCs was confirmed by Western blot analysis. Unlike cord blood (CB) HSPCs, human iPS-HSPCs do not bind to E-selectin (right panel). This is a representative experiment of n=3 independent experiments. The gates were set based on the isotype controls after compensation. FIG. 7B shows flow cytometric analysis of iPS-HSPCs treated with rhFTVI shows that the cells were appropriately fucosylated and gained HECA-452 reactivity on their surface (left panel). Western blot analysis revealed the binding of E-selectin following rhFTVI treatment to iPS-HSPCs whole cell lysates (upper right panel) and immunoprecipitations of CD43 and CD44 from iPS-HSPC lysates (lower right panel). FIG. 7C shows multipotent clonal behavior of iPS-HSPCs. CFU assay was performed on iPS-HSPCs either treated with rhFTVI (+) or with buffer alone (−). On day 21 of culture, total colonies were enumerated for erythroid burst-forming units (BFU-Es), granulocyte-macrophage colony-forming units (CFU-GMs), and granulocyte-erythroid-megakaryocyte-macrophage colony-forming units (CFU-GEMMs). Results show that treatment with rhFTVI (+) did not affect the multipotent clonal behavior of iPS-HSPCs.

### DETAILED DESCRIPTION OF THE INVENTION

### Definitions

As used herein the term “isolated” is meant to describe a compound of interest (e.g., either a polynucleotide or a polypeptide) that is in an environment different from that in which the compound naturally occurs e.g. separated from its natural milieu such as by concentrating a peptide to a concentration at which it is not found in nature. “Isolated” is meant to include compounds that are within samples that are significantly enriched for the compound of interest and/or in which the compound of interest is partially or significantly purified. “Significantly” means statistically significantly greater.

As used herein, the term “polypeptide” refers to a chain of amino acids of any length, regardless of modification (e.g., phosphorylation or glycosylation).

As used herein, a “variant” polypeptide contains at least one amino acid sequence alteration as compared to the amino acid sequence of the corresponding wild-type polypeptide.

As used herein, a “vector” is a replicon, such as a plasmid, phage, or cosmid, into which another DNA segment may be inserted so as to bring about the replication of the inserted segment. The vectors described herein can be expression vectors.

As used herein, an “expression vector” is a vector that includes one or more expression control sequences

As used herein, an “expression control sequence” is a DNA sequence that controls and regulates the transcription and/or translation of another DNA sequence.

As used herein, “operably linked” means incorporated into a genetic construct so that expression control sequences effectively control expression of a coding sequence of interest.

As used herein, “transformed” and “transfected” encompass the introduction of a nucleic acid (e.g., a vector) into a cell by a number of techniques known in the art.

Recitation of ranges of values herein are merely intended to serve as a shorthand method of referring individually to each separate value falling within the range, unless otherwise indicated herein, and each separate value is incorporated into the specification as if it were individually recited herein.

A “polyhistidine-tag” as used herein refers to an amino acid motif in proteins that consists of at least six histidine (His) residues, often at the N- or C-terminus of the protein

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### II. Compositions

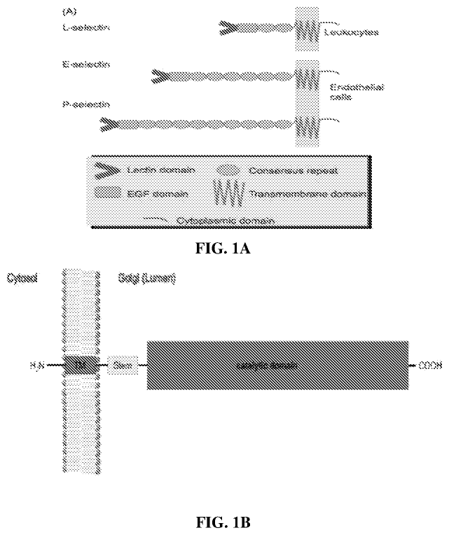

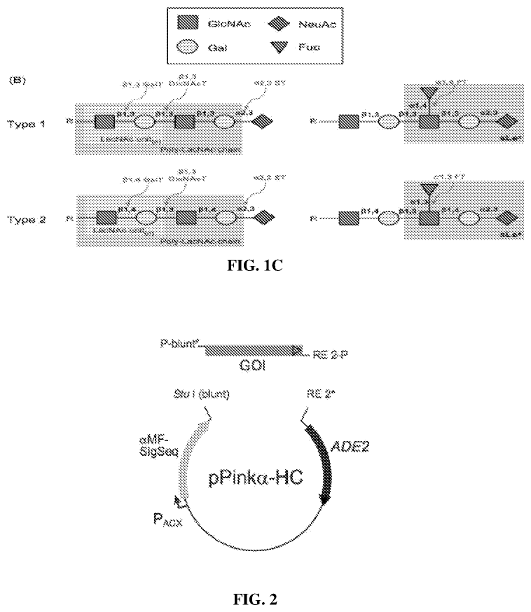

The disclosed compositions include recombinantly produced GT enzymes, which include the luminal catalytically active fragment of the GT enzyme. The compositions preferably do not include the full polypeptide of the GT enzyme. See FIG. 1B. The enzymes are purified from the Pichia pastoris expression system disclosed herein. The enzyme compositions are preferably lyophilized and more preferably, contain 0% glycerol or at least 50% glycerol.

A. Glycosyltransferase Enzymes

Glycosyltransferases catalyze the transfer of sugar residues from nucleotide-sugars to specific acceptor (carbohydrates or glycan chains) according to the following general equation:

Nucleotide-sugar+Acceptor=Sugar-Acceptor+Nucleotide GTs are type II transmembrane glycoproteins consisting of a short amino-terminal cytoplasmic tail, transmembrane region, an extended stem region and a large carboxy-terminal catalytic domain which is oriented to the lumen of the ER or Golgi apparatus. The display of glycan structures on selectin ligands requires the expression and activity of various Glycosyltransferases (GT), including the action of α1,3- or α1,4-fucosyltransferases (FUT), α2,3-sialyltransferases (ST), β1,4-galactosyltransferases (GalT), and β1,6-N-acetylglucosaminyltransferases (GlcNAcT).

Accordingly, the compositions include recombinantly produced α1,3- or α1,4-fucosyltransferases (FUT), α2,3-sialyltransferases (ST), β1,4-galactosyltransferases (GalT), and β1,6-N-acetylglucosaminyltransferases (GlcNAcT), for example, produced as exemplified herein for FUT6.

Basically, each enzyme catalyzes only one of several sugar nucleotide substrates (including UDP-Galactose (Gal), UDP-Glucose (Glc), UDP-N-acetylgalactosamine (GalNAc), UDP-N-acetylglucosamine (GlcNAc), GDP-fucose (Fuc), GDP-mannose (Man), UDP-xylose (Xyl), or CMP-sialic acid (SA)). In addition, the acceptor for each GT is quite specific with few exceptions and only capable of forming one particular glycosidic bond (i.e., either an α or β anomer).

O-Glycan synthesis starts by the action of GalNAcT enzymes that transfer GalNAc residue to a serine or the threonine on the polypeptide. Then, by the addition of Gal in a core 2 branching is formed by the addition of GlcNAc from C2GlcNAcT enzyme action in β1-6 linkage. These branches are then extended by the addition of GlcNAc and Gal alternately to form polylactosamine side chains. In this step, the terminal cap or sialylated Lewis epitope is the most important to determine if the molecule will play a role in cell adhesion selectin ligand activity; this is determined by two enzymes σ1,3- or α1,4-fucosyltransferases (FUTs) and α2,3-sialyltransferases (STs). This pathway finally ends up with the formation of the O-linked core two based tetrasaccharide sialyl Lewis^{x/a}, which is composed of sialic acid, Gal, GlcNAc and Fuc. Generally, from STs family ST3Gal-III create sLe^{a} by acting on type 1 Lactosamine while ST3Gal-IV and ST3Gal-VI give sLe^{x} that mainly act on type 2 Lactosamines. On the other hand, α1,3 fucosylation plays a role for E-selectin ligand creation. This is outlined above in FIG. 1C.

Fucosyltransferases (FUTs)

FUTs use GDP-fucose as donor substrate and as a result it plays a significant role in fucosylated glycans.

The fucosyltransferase family share the same structural characteristics (FIG. 1B) including a type 2 transmembrane Golgi-anchored proteins containing an N-terminal cytoplasmic tail, a transmembrane region, and an extended stem region followed by a large globular C-terminal catalytic domain facing the Golgi lumen. This family consists of 13 enzymes that have been identified in the human genome and classified either according to the type of linkage, based on the site of fucose addition, into α1,2, α1,3/4, α1,6, and O-FUTs or according to sequence analysis and their similarities. All FUTs enzymes bind GDP-fucose that imply they have the same consensus sequence in donor substrate binding (Lys300). FUTs add fucose on sialylated precursors, so they catalyzed the final step in glycoconjugate synthesis resulting in sLe^{x/a} expression. They transfer the fucose residue from GDP-fucose (donor substrate) to GlcNAc in Gal-GlcNAc-sequences (acceptor substrate) in α1,3/4 linkage to form sLe^{x/a} that could bind to counterpart selectins.

In mammalian cells, there are six α1,3/4 FUTs, FUT3-7 and FUT9 (or Fuc-TIII-VII and Fuc-TIX), all of which have a1,3 activity, but FUT3 and FUTS also has a1,4 activity.

A preferred FUT is FUT6, referred to herein as FTVI, interchangeably.

Consensus sequences for human α-1,3-Fucosyltransferase (FUT6) are known in the art. See, for example, UniProtKB—P51993 (FUT6_HUMAN), which provides a consensus amino acid sequence, variants and alternate isoforms thereof, and accession numbers for mRNA and genomic sequences.

A consensus amino acid sequence for human FIJT6 is

(SEQ ID NO: 1; GenBank: M98825.1; UniProtKB -
P51993 (FUT6_HUMAN))
MDPLGPAKPQWSWRCCLTTLLFQLLMAVCFFSYLRVSQDDPTVYPNGSRF
PDSTGTPAHSIPLILLWTWPFNKPIALPRCSEMVPGTADCNITADRKVYP
QADAVIVHHREVMYNPSAQLPRSPRRQGQRWIWFSMESPSHCWQLKAMDG
YFNLTMSYRSDSDIFTPYGWLEPWSGQPAHPPLNLSAKTELVAWAVSNWG
PNSARVRYYQSLQAHLKVDVYGRSHKPLPQGTMMETLSRYKFYLAFENSL
HPDYITEKLWRNALEAWAVPVVLGPSRSNYERFLPPDAFIHVDDFQSPKD
LARYLQELDKDHARYLSYFRWRETLRPRSFSWALAFCKACWKLQEESRYQ
TRGIAAWFT

A nucleic acid sequence (cDNA) encoding SEQ ID No:1 is

(SEQ ID NO: 1; GenBank: M98825.1)
CAGATACTCTGACCCATGGATCCCCTGGGCCCGGCCAAGCCACAGTGGTC
GTGGCGCTGCTGTCTGACCACGCTGCTGTTTCAGCTGCTGATGGCTGTGT
GTTTCTTCTCCTATCTGCGTGTGTCTCAAGACGATCCCACTGTGTACCCT
AATGGGTCCCGCTTCCCAGACAGCACAGGGACCCCCGCCCACTCCATCCC
CCTGATCCTGCTGTGGACGTGGCCTTTTAACAAACCCATAGCTCTGCCCC
GCTGCTCAGAGATGTGTCCTGGCACGGCTGACTGCAACATCACTGCCGAC
CGCAAGGTGTATCCACAGGCAGACGCGGTCATCGTGCACCACCGAGAGGT
CATGTACAACCCCAGTGCCCAGCTCCCACGCTCCCCGAGGCGGCAGGGGC
AGCGATGGATCTGGTTCAGCATGGAGTCCCCAAGCCACTGCTGGCAGCTG
AAAGCCATGGACGGATACTTCAATCTCACCATGTCCTACCGCAGCGACTC
CGACATCTTCACGCCCTACGGCTGGCTGGAGCCGTGGTCCGGCCAGCCTG
CCCACCCACCGCTCAACCTCTCGGCCAGACCGAGCTGGTGGCCTGGGCAG
TGTCCAACTGGGGGCCAAACTCCGCCAGGGTGCGCTACTACCAGAGCCTG
CAGGCCCATCTCAAGGTGGACGTGTACGGACGCTCCCACAAGCCCCTGCC
CCAGGGAACCATGATGGAGACGCTGTCCCGGTACAAGTTCTATCTGGCCT
TCGAGAACTCCTTGCACCCCGACTACATCACCGAGAAGCTGTGGAGGAAC
GCCCTGGAGGCCTGGGCCGTGCCCGTGGTGCTGGGCCCCAGCAGAAGCAA
CTACGAGAGGTTCCTGCCGCCCGACGCCTTCATCCACGTGGACGACTTCC
AGAGCCCCAAGGACCTGGCCCGGTACCTGCAGGAGCTGGACAAGGACCAC
GCCCGCTACCTGAGCTACTTTCGCTGGCGGGAGACGCTGCGGCCTCGCTC
CTTCAGCTGGGCACTCGCTTTCTGCAAGGCCTGCTGGAAACTGCAGGAGG
AATCCAGGTACCAGACACGCGGCATAGCGGCTTGGTTCACCTGAGAGGCC
CGGCATGGGGCCTGGGCTGCCAGGG

Two transcript variants encoding the same protein have been found for this gene.

FUT6 has a preference for N-Acetyllactosamine (Galβ1-4GlcNAc) and also good specificity towards 3′-Sialyl-N-acetyllactosamine, (NeuAcα2-3Galβ1-4GlcNAc). The human FUT6 gene is located on chromosome 19p13.3 and it has six exons. It encodes for a 359 amino acids peptide including N-terminal region that is composed of the cytoplasmic sequence, signal-anchor for type II membrane sequence while the C-terminal region consist of luminal sequence that contains catalytic domain (composed of 325 aa), the third part between the membrane-spanning region and catalytic domain is a region called stem region. FUT6 exceeds the size of FUT3 by 15 amino acids. The N-terminal region may not be required for activity, so it can be deleted without any effect on enzyme activity while any change in C-terminal region may result in the production of an inactive enzyme.

Sialyltransferases

Sialyltransferases (ST) belong to glycosyltransferase family 29 which include enzymes with a number of known activities; sialyltransferase (EC 2.4.99), beta-galactosamide alpha-2,6-sialyltransferase (EC 2.4.99.1), alpha-N-acetylgalactosaminide alpha-2,6-sialyltransferase (EC 2.4.99.3), beta-galactoside alpha-2,3-sialyltransferase (EC 2.4.99.4), N-acetyllactosaminide alpha-2,3-sialyltransferase (EC 2.4.99.6), alpha-N-acetyl-neuraminide alpha-2,8-sialyltransferase (EC 2.4.99.8); lactosylceramide alpha-2,3-sialyltransferase (EC 2.4.99.9). These enzymes use a nucleotide monophosphosugar as the donor (CMP-NeuA) instead of a nucleotide diphosphosugar. Sialyltransferases can be distinguished on the basis of the acceptor structure on which they act and on the type of sugar linkage they form. Some sialyltransferases adds sialic acid with an alpha-2,3 linkage to galactose, while others sialyltransferases add sialic acid with an alpha-2,6 linkage to galactose or N-acetylgalactosamine A peculiar type of sialyltransferases add sialic acid to other sialic acid units with an alpha-2,8 linkage, forming polysialic acid. For example, α-2,3-sialyltransferase ST3 enzymes transfer sialic acids to C-3 of galactose residue in acceptor glycans.

Mammalian STs are Type II transmembrane glycoproteins with a short 3-11 amino acid NH2-terminal cytoplasmic domain, which is not essential for catalytic activity, a 16-20 amino acid transmembrane (signal anchor) domain, a 30-200 amino acid extended stem region, followed by large 300-350 residue COOH-terminal catalytic domain.

Galactosyltransferases

Galactosyltransferas catalyzes the transfer of galactose. Glycosyltransferase family includes enzymes with a number of known activities; N-acetyllactosaminide beta-1,3-N-acetylglucosaminyltransferase (EC 2.4.1.149); beta-1,3-galactosyltransferase (EC 2.4.1); fucose-specific beta-1,3-N-acetylglucosaminyltransferase (EC 2.4.1); globotriosylceramide beta-1,3-GalNAc transferase (EC 2.4.1.79).

B. Enzyme Compositions

Enzyme composition comprises purified recombinant GT enzyme in an acceptable buffer, comprising at up to 50% glycerol as a stabilizer. In some preferred embodiments, the composition comprises 0% glycerol as a stabilizer and in some embodiments a divalent a cation, for example manganese. In other embodiments the composition comprises at least 50% glycerol as a stabilizer. In a particularly preferred embodiment, the enzyme composition is lyophilized.

### III. Methods of Making

Recombinant GT for example, human FUT6, β-1,4-galactosyltransferase and α-2,6-sialyltransferse have been expressed and purified in several eukaryotic systems including CHO cells, insect cells, and yeast expression systems (Malissard et al., 2000). Although all these systems produced functional rhFUT6, several disadvantages exist related to the ease of expression and cost. Typically, such purification procedures require synthetic columns with multiple steps for the purification of proteins and are associated with high costs for materials especially for expression in mammalian systems (i.e. CHO cells). For example, the disclosed methods do not require a purification technique which involved making guanine diphosphate (GDP-hexanolamine column.

The methods disclosed herein provide a more simplified, more practical procedure to produce functional enzymatically active GT in a single step purification using IMAC (immobilized metal affinity chromatography), which is cost effective for the preparation of large-scale proteins.

To this end, following the expression of rhFTVI in Pichia pastoris, the samples (supernatant and cell lysates) are concentrated and purified using a single step nickel column; the samples were then dialyzed and the rhFTVI is characterized. One advantage of intracellular expression shown with the disclosed expression system is much smaller sample volume (as lysate), typically 40 fold reduction compared with that of secreted expression (as supernatant), which contributes to the simpler/easier purification step here. Processing large quantity of supernatant could be time-consuming, labor-intensive or require a specific instrument e.g. tangential flow filtration system.

In order to introduce the GT genes into the Pichia pastoris expression system, the cytoplasmic tail and transmembrane region are replaced with a cleavable signal sequence.

Recombinant GTs, especially fucosyltransferases, may be produced in different expression systems. Although relatively practical and simple with large potential yields, bacterial expression systems do not result in enzymatically active GTs likely due to the absence of glycosylation machinery required for enzymatic activity i.e. N-glycosylation. The yeast, Pichia Pastoris, expression system may be used to express many glycosyltransferases involved in the biosynthesis of N- and O-linked oligosaccharides. This is summarized in Table 1. In summary, the choice between expression systems depends on many factors, the nature and use of the recombinant protein, and the related production costs. Yeast expression systems combine the ease, simplicity and cost effectiveness of bacterial systems to the high quality post-translationally modified protein of mammalian systems.

TABLE 1
The expression of recombinant GTs
in different expression systems.

| Glycosyltransferases | Expression system |
|---|---|
| β-1,4-Galactosyltransferase | Pichia pastoris |
| | Insect cells |
| α-2,6-Sialytranseferase | Pichia pastoris |
| α-1,3-Fucosyltransferase 3 | Baby Hamster |
| | Kidney cells |
| | IBHK-21B) |
| | Pichia pastoris |
| | CHO cells |
| | insect cells |
| α-1,3-Fucosyltransferase 5 | Insect and |
| | Mammalian systems |
| α-1,3-Fucosyltransferase 6 | Pichia pastoris |
| | Insect cells |
| | CHO cells |
| α-1,3-Fucosyltransferase 7 | Insect cells |
| | Yeast cells |
| | CHO and COS-7 |
| α-1,3-Fucosyltransferase 9 | Insect cells |
| | Hela Cells |
| | E. coli and |
| | mammalian systems |
| α-2,3 sialyltransferase | Yeast cells |
| ST3GalIII | |
| α-2,6 sialyltransferase | Yeast cells |
| α-2,6 sialyltransferase | Yeast cells |

The methods for recombinantly producing enzymatically active GTs relies on a yeast expression system, preferably, a Pichia pastoris, expression system and more preferably, an expression system that uses the Pichia pastoris, more preferably, a Pichia pastoris stain with an ade2 deletion.

This strain is and ADE2 auxotrophs that is unable to grow in the absence of adenine because of full deletion of the ADE2 gene and part of its promoter. The ADE2 gene encodes phosphoribosylaminoimidazole carboxylase, which catalyzes the sixth step in the de novo biosynthesis of purine nucleotides.

The method includes genetically engineering a host organism to express a GT enzyme, preferably, the luminal domain of the GT enzyme, comprising its catalytic domain. The host organism is Pichia pastoris more preferably, a Pichia pastoris stain with an ade2 deletion.

The method includes introducing into the Pichia pastoris host, a vector containing gene encoding the catalytic domain of the GT enzyme, operably linked to one or more expression control sequences and a Pichia pastoris secretion signal, preferably, the α-mating factor. The vector preferably comprises a 6×Histidine (His)-tag added to the N-terminus of the gene encoding the GT enzyme. A particularly preferred GT transferase enzyme is human alpha-(1,3)-fucosyltransferase, more preferably, human alpha-(1,3)-fucosyltransferase 6 (FUT6). The method further comprises purifying the recombinantly expressed GT enzyme from the host cells, immobilized metal affinity chromatography (IMAC) as a preferred purification method.

Vectors for Recombinant Expression of GTs

The construct design focuses on the selection of promotors (induced or constitutive) and whether the target protein will be expressed intracellularly or extracellularly (i.e. released into the supernatant/media)

Nucleic acids encoding the catalytic domain of the GT of interest, can be inserted into vectors for expression in cells. As used herein, a “vector” is a replicon, such as a plasmid, phage, or cosmid, into which another DNA segment may be inserted so as to bring about the replication of the inserted segment. Vectors can be expression vectors. An “expression vector” is a vector that includes one or more expression control sequences, and an “expression control sequence” is a DNA sequence that controls and regulates the transcription and/or translation of another DNA sequence.

Nucleic acids in vectors can be operably linked to one or more expression control sequences. As used herein, “operably linked” means incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

Suitable expression vectors include, without limitation, plasmids and viral vectors derived from, for example, bacteriophage, baculoviruses, tobacco mosaic virus, herpes viruses, cytomegalovirus, retroviruses, vaccinia viruses, adenoviruses, and adeno-associated viruses. Numerous vectors and expression systems are commercially available from such corporations as Novagen (Madison, Wis.), Clontech (Palo Alto, Calif.), Stratagene (La Jolla, Calif.), and Invitrogen Life Technologies (Carlsbad, Calif.).

An expression vector can include a tag sequence. Tag sequences, are typically expressed as a fusion with the encoded polypeptide. Such tags can be inserted anywhere within the polypeptide including at either the carboxyl or amino terminus. Examples of useful tags include, but are not limited to, HIS-TAG, green fluorescent protein (GFP), glutathione S-transferase (GST), polyhistidine, c-myc, hemagglutinin, Flag™ tag (Kodak, New Haven, Conn.), maltose E binding protein and protein A. A preferred tag is the HIS-TAG. The DNA sequence specifying a string of six to nine histidine residues is preferably used in vectors for production of recombinant proteins. The result is expression of a recombinant protein with a 6×His or poly-His-tag fused to its N- or C-terminus.

In some preferred embodiments, the vector can include a protease cleave site that allows cleavage of the tag, following purification. An example is the tobacco etch virus (TEV) protease cleavage site for removing the tag from the recombinant protein.

Isolated nucleic acid molecules encoding GT polypeptides can be produced by standard techniques, including, without limitation, common molecular cloning and chemical nucleic acid synthesis techniques. For example, polymerase chain reaction (PCR) techniques can be used to obtain an isolated nucleic acid encoding a variant costimulatory polypeptide. PCR is a technique in which target nucleic acids are enzymatically amplified. Typically, sequence information from the ends of the region of interest or beyond can be employed to design oligonucleotide primers that are identical in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. General PCR techniques are described, for example in PCR Primer: A Laboratory Manual, ed. by Dieffenbach and Dveksler, Cold Spring Harbor Laboratory Press, 1995. When using RNA as a source of template, reverse transcriptase can be used to synthesize a complementary DNA (cDNA) strand. Ligase chain reaction, strand displacement amplification, self-sustained sequence replication or nucleic acid sequence-based amplification also can be used to obtain isolated nucleic acids. See, for example, Lewis (1992) Genetic Engineering News 12:1; Guatelli et al. (1990) Proc. Natl. Acad. Sci. USA 87:1874-1878; and Weiss (1991) Science 254:1292-1293.

Isolated nucleic acids can be chemically synthesized, either as a single nucleic acid molecule or as a series of oligonucleotides (e.g., using phosphoramidite technology for automated DNA synthesis in the 3′ to 5′ direction). For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase can be used to extend the oligonucleotides, resulting in a single, double-stranded nucleic acid molecule per oligonucleotide pair, which then can be ligated into a vector.

Pichia pastoris Transformation and Culture

Vectors containing nucleic acids to be expressed can be transferred into the Pichia pastoris host cells. As used herein, “transformed” and “transfected” encompass the introduction of a nucleic acid molecule (e.g., a vector) into a cell by one of a number of techniques. Although not limited to a particular technique, a number of these techniques are well established within the art. To recombinantly produce a GT enzyme, a nucleic acid containing a nucleotide sequence encoding the polypeptide (preferably only the catalytic domain of the GT enzyme can be used to transform, transduce, or transfect Pichia pastoris host cells. In general, nucleic acid constructs include a regulatory sequence operably linked to a nucleotide sequence encoding a GT enzyme catalytic domain. Regulatory sequences (also referred to herein as expression control sequences) typically do not encode a gene product, but instead affect the expression of the nucleic acid sequences to which they are operably linked.

A number of viral-based expression systems disclosed as useful in eukaryotic systems can be utilized to express enzymatically active GT enzymes. Viral based expression systems are well known in the art and include, but are not limited to, baculoviral, SV40, retroviral, or vaccinia based viral vectors.

Following introduction of an expression vector by electroporation, lipofection, calcium phosphate, or calcium chloride co-precipitation, DEAE dextran, or other suitable transfection method, stable cell lines can be selected as exemplified in the examples. The transfected cells are cultured such that the polypeptide of interest is expressed.

A Pichia pastoris expression system is preferred for recombinant expression of GTs as it has the ability to overcome the hyperglycosylation of recombinant proteins, the possibility to secrete soluble forms of proteins and may be used to produce proteins for many therapeutic purposes. The cultivation of yeast cells may require its own growth media either in shaker flasks or fed-batch cultivation with fermentation. There are two phases for methylotrophic P. pastoris. It begins with the growth phase to produce high growth rates (>100 g/l on glycerol media) and then production of target protein at low growth rates by induction with methanol as carbon source at an optimum temperature.

An exemplary culture protocol includes selecting single colonies of Pichia pastoris yeast that express recombinant FUT6, and inoculating the colonies in an appropriate buffered medium, for example, a complex glycerol or methanol medium (BMGY or BMMY), composed of 1% yeast extract (BD), 2% peptone (BD), 100-mM potassium phosphate, pH=6.0 (Fisher Scientific), 1.34% YNB (Sigma), 0.0004% biotin (Sigma) and 1% glycerol (Sigma; BMGY medium) or 0.5% methanol (VWR; BMMY medium). The cells are grown first in BMGY media for 1-2 and then transferred BMGY medium and cultured for another day under the same conditions. The cells further cultured in BMGY medium and cultured for two days. The cells are then pelleted in a sterile centrifuge bottle re-suspended in BMMY medium to induce expression. This is followed by cell culture (at 30° C.) in a shaking incubator for an additional seven days with the addition of 0.5% methanol daily. Subsequently, the cells are harvested and placed in a suitable lysis buffer on 8th day of induction, cells were harvested by centrifugation at 3000-rpm for 10-min and re-suspended in 200-mL of lysis buffer, for example, 100-mM potassium phosphate (Fisher Scientific), 500-mM NaCl (Fisher Scientific), 10-mM MnCl2 (Fisher Scientific), 2.5-mM imidazole 1-mM PMSF (Alexis) and EDTA free protease inhibitor cocktail tablet (Roche, UK) pH 7.8.

Purification of Recombinantly Produced GT

The GT polypeptide can be recovered from, for example, the cell culture supernatant and/or from lysed cells. Preferably, the polypeptide is recovered from lysed cells. The expressed protein is preferably purified based on HIS tag it expresses. Expressed His-tagged proteins can be purified and detected easily because the string of histidine residues binds to several types of immobilized metal ions, including nickel, cobalt and copper, under specific buffer conditions. In addition, anti-His-tag antibodies are commercially available for use in assay methods involving His-tagged proteins. In either case, the tag provides a means of specifically purifying or detecting the recombinant protein without a protein-specific antibody or probe.

In a particularly preferred embodiment, the protein purification step relies on immobilized metal affinity chromatography. Supports such as beaded agarose or magnetic particles can be derivatized with chelating groups to immobilize the desired metal ions, which then function as ligands for binding and purification of biomolecules of interest. This basis for affinity purification is known as immobilized metal affinity chromatography (IMAC). The chelators most commonly used as ligands for IMAC are nitrilotriacetic acid (NTA) and iminodiacetic acid (IDA). Once IDA-agarose or NTA-agarose resin is prepared, it can be “loaded” with the desired divalent metal (e.g., Ni, Co, Cu, and Fe). Using nickel as the example metal, the resulting affinity support is usually called Ni-chelate, Ni-IDA or Ni-NTA resin. Nickel or cobalt metals immobilized by NTA-chelation chemistry are preferred. In addition, different varieties of agarose resin provide supports that are ideal for His-tagged protein purification at very small scales (96-well filter plates) or large scales (series of chromatography cartridges in an FPLC system). When packed into suitable columns or cartridges, resins such as Ni-NTA Superflow Agarose provide for purification of 1 to 80 milligrams of His-tagged protein per milliliter of agarose beads.

Poly-His tags bind best to IMAC resins in near-neutral buffer conditions (physiologic pH and ionic strength). A typical binding/wash buffer consists of Tris-buffer saline (TBS) pH 7.2, containing 10-25 mM imidazole. The low-concentration of imidazole helps to prevent nonspecific binding of endogenous proteins that have histidine clusters.

Elution and recovery of captured His-tagged protein from an IMAC column is accomplished by using a high concentration of imidazole (at least 200 mM), low pH (e.g., 0.1 M glycine-HCl, pH 2.5) or an excess of strong chelators (e.g., EDTA). Imidazole is the preferred elution agent. Imidazole competes with the his-tag for binding to the metal-charged resin and thus is used for elution of the protein from an IMAC column. Typically, a low concentration of imidazole is added to both binding and wash buffers to interfere with the weak binding of other proteins and to elute any proteins that weakly bind. His-tagged protein is then eluted with a higher concentration of imidazole.

### IV. Methods of Using

Most intravenous therapeutic adult stem cells have limited engraftment efficiency to their target tissue due to lack of key homing molecules. In such cases, these therapeutic cells require additional methods to improve homing.

Hematopoietic stem cell transplantation (HSCT) is the most common cell-based therapy currently used in clinical practice. It is offered to patients with life-threatening blood disorders and hematological malignancies. Currently, the only sources for transplantable hematopoietic stem and progenitor stem cell (HSPCs) are bone marrow (BM), umbilical cord blood (CB), or mobilized peripheral blood (Amos and Gordon, 1995; Haspel and Miller, 2008). The number of isolated HSPCs from those sources is very limited in supply and only one-third of the patients find HLA-matched donor cells (Choi et al., 2009; Szabo et al., 2010; Park et al., 2013). Direct differentiation of HSPCs from pluripotent sources such induced pluripotent stem cells (iPS) theoretically offers an unlimited source of allo-/autologous HSPCs for transplantation therapies. In vitro studies have shown that iPS-derived HSPCs (iPS-HSPCs) behave much like somatic HSPCs exhibiting robust clonal proliferation and multilineage hematopoietic capacity (Chadwick et al., 2003; Vodyanik et al., 2005; Wang et al., 2005; Bhatia, 2007; Choi et al., 2009; Szabo et al., 2010; Tolar et al., 2011; Park et al., 2013). Thus, iPS hold great promise since they are amenable to large-scale production and can overcome the challenge of finding immune-compatible donors. Nonetheless, the utilization of iPS-HSPCs in HSCT is limited by the relative scarcity of finding them in the bone marrow following transplantation (Vodyanik et al., 2005; Ji et al., 2008; Ledran et al., 2008; Amabile et al., 2013; Suzuki et al., 2013; Dou et al., 2016).

Glycan-Engineering methods could be used to create glycan structures such as sLe^{x} and sLe^{a} on the cell surface in order to guide the delivery of cells to their target tissues where specific selectins are expressed. The recombinantly produced GT disclosed herein can be used ex vivo to create glycan structures such as sLe^{x} and sLe^{a} on the surface of a cell in need thereof.

Cells that can benefit from the ex vivo treatment disclosed herein include any cells used for cell therapy, for example, hematopoietic stem cells, neural stem cells, induced pluripotent stem cells, skeletal myoblasts, bone marrow cells, circulating blood-derived progenitor cells, endometrial mesenchymal stem cells, adult testis pluripotent stem cells, mesothelial cells, adipose-derived stromal cells, embryonic cells, induced pluripotent stem cells, and bone marrow.

The method includes contacting a cell with the disclosed compositions comprising purified recombinant GT enzyme and a substrate (nucleotide sugar) for the GT enzyme for an effective amount of time and culture conditions for the GT enzyme to catalyze transfer of substrate onto an acceptor site at the surface of the cell. The sugar substrates are selected from the group consisting of UDP-Galactose (Gal), UDP-Glucose (Glc), UDP-N-acetylgalactosamine (GalNAc), UDP-N-acetylglucosamine (GlcNAc), GDP-fucose (Fuc), GDP-mannose (Man), UDP-xylose (Xyl), or CMP-sialic acid (SA)), depending on the GT enzyme in the reaction mixture. The composition in preferred embodiments does not include glycerol as a stabilizer or it includes at least 50% glycerol. In a particularly preferred embodiment, the enzyme compositions include 0% glycerol and Mn added in concentrations between 3-4 Mm.

The present invention will be further understood by reference to the following non-limiting examples.

### EXAMPLES

### Material and Methods

### Construction FUT6 Recombinant Vector and Transformation

### OF Pichia Pastoris

Briefly, a pPink-aHC vector was used (Invitrogen) to integrate the human FUT6 cDNA encoding amino acid 35-359 of the FUT6 protein sequence that omits the cytoplasmic and transmembrane regions of full length human FUT6, and encompassed the entire catalytic domain of the enzyme. The vector was propagated in E. coli strain TOP 10F (Invitrogen). The recovered DNA was linearized with restriction enzyme and then the digested DNA were used to transform Pichia Pastoris strains according to the manufacturer's instructions (Invitrogen). Stable transformants were selected on minimal medium agar plates (MD plates) for further processing.

Construction of Recombinant Vector and Transform to E. coli Cells

A) The cDNA encoding soluble form of human FUT6 were generated by PCR with FUT6 primers and the FUT6 contained six histidine (His-tag) at N-terminus and must have a phosphorylated 5 ‘ blunt end (adding an Mly I site) and a 3’ overhang after the stop codon that is compatible to the restriction enzyme used to linearize pPinka-HC (Kpn I). FUT6 lack any internal restriction site for Mly I and the restriction enzyme used.

B) pPink-aHC vector was used from (Invitrogen) to subclone the human FUT6 open reading frame (ORF) downstream of the α-mating factor pre-sequence. The PichiaPink vectors contain the ampicillin resistance gene to allow selection of the plasmid using ampicillin. About 0.5 μg/μl vector linearized by double digestion with 10 units/μL Stu I restriction enzyme (created a blunt end) and 10 units/μL, Kpn I restriction enzyme in the multiple cloning site downstream of the Stu I site that does not cut within FUT6. It was incubated for 2 hours overnight at 37 C° then added Calf Intestinal Alkaline Phosphatase (CIAP) (1 unit/μL) to dephosphorylate the vector and then the proper digestion by a gel was checked.

C) Ligation of Vector with Human FUT6 cDNA

A ligation reaction was established in a 0.5 mL micro-centrifuge tube by gently mixing 2 uL of 5× ligase buffer, 0.5 μL of T4 DNA ligase 1 of 20 ng/uL pPinka-HC (FIG. 2A) and 1 μL, of 20 ng/μL of FUT6 gene and then centrifuged briefly, and incubated the mix at 25 C.° for 1-2 hours, and/or at 16 C° overnight.

Briefly, the pPink-aHC vector was used (Invitrogen) to integrate the human FTVI cDNA encoding amino acid 35-359 of the FTVI protein sequence that omits the cytoplasmic and transmembrane regions of full length human FTVI and encompassed the entire catalytic domain of the enzyme. A human FTVI luminal domain sequence was codon-optimized with JCat software, synthesized, and amplified with the primers AGAGTTTCTCAAGACGACCCAACTGTTTAC and TGGTACCAGTGAACCAAGCAGCGATACCTCTAGT. The obtained fragment was digested with Kpnl and ligated together with a pPink-aHC fragment digested with Stul and Kpnl to construct pPink-aHC-hFTVIlum. In order to include 6×Histidine-tag on the N-terminus of the FTVI ORF, a PCR reaction was conducted with the primers GGCATCATCACCATCATCA TGGTAGAGTTTCTCAAGACGACCCA and CTACCATGATGATGGTG ATGATGCCTTTTCTCGAGAGATACCCCTTC.

D) Transformation to E. coli:

This step was to analyze the transformants for the presence and proper orientation of FUT6 gene. pPinka-HC contains Ampicillin resistance gene, following the pichiapink protocol for transformation step by electroporation with 0.1 cm cuvette, and plating the E. coli cells in LB agar contain ampicillin including, one plate for cells only and one for vector only as a control. To identify the correct clone, 6-8 colonies per plate were picked up and positive colonies by PCR and sequencing were analyzed. The colony was purified and a glycerol stock was made for long term storage. The plasmids were isolated from E. coli by using PureLink Quick Plasmid Miniprep Kit to introduce them to Pichia strains.

### Transformation of Pichia Pastoris Strain

Pichia Pink Plasmid DNA was purified and linearized before transformation and selection in PichiaPink strains from (invitrogen). First, wild-type ade2 knockout Pichia was prepared by placing it in YPD media and then 5-10 μg of linearized plasmid was transformed by electroporation. The ade2 knockout renders the PichiaPink strain an adenine auxotroph, which needs an external adenine source for growth and the pichiaPink vector had this ade2 gene. These cells are unable to grow on minimal medium or adenine dropout medium unless it contains recombinant vector. The “vector only” and “cells only” controls were included to evaluate the experiment. The positive transformants were identified by direct PCR screening. The transformants were plated into yeast agar plate minimal media called synthetic dropout that lack only one nutrient as Adenine and another nutritional agar YPD contained 1% yeast extract, 2% bactopeptone and 2% Dextrose. Then incubated for 2-3 days at 30° C.

### FUT6 Expression in Pichia Pastoris

By using BMGY and BMMY (buffered complex glycerol or methanol medium), for expression of FUT6. These media are buffered with phosphate buffer and contain yeast extract and peptone to stabilize secreted proteins and prevent or decrease proteolysis of secreted proteins. All expression is done at 30° C., in a shaking incubator at 200 rpm. Buffered Glycerol-complex Medium and Buffered Methanol-complex Medium (1 liter) composed of 1% yeast extract, 2% peptone, 100 mM potassium phosphate (pH 6.0), 1.34% YNB, 0.0004% biotin and 1% glycerol or 0.5% methanol.

The growing and expression required around 12 days. For FTVI expression, single colonies of Pichia pastoris yeast that express recombinant human FTVI were inoculated in 1-L baffled flasks containing 100-mL buffered complex glycerol or methanol medium (BMGY or BMMY), composed of 1% yeast extract (BD), 2% peptone (BD), 100-mM potassium phosphate, pH=6.0 (Fisher Scientific), 1.34% YNB (Sigma), 0.0004% biotin (Sigma) and 1% glycerol (Sigma; BMGY medium) or 0.5% methanol (VWR; BMMY medium). Briefly, the cells were grown first in 100-mL of BMGY media for 1-2 days at 30° C. in a shaking incubator set at 200-rpm. After the incubation the cells were transferred to a 1 L of BMGY incubate another day with the same condition. Then in the following day, the cells were divided into five/2 L flasks of BMGY for two days in the same condition (30° C. and 200 rpm). The approximate number of cells in a culture was determined with a spectrophotometer by measuring the optical density (OD) at 600-nm. The cells were then pelleted in a sterile centrifuge bottle at 3000×g for 15-min and re-suspended the cell in two/1-L of BMMY medium to induce expression and then cultured at 30° C. in a shaking incubator for an additional seven days with the addition of 0.5% methanol daily. At day 8 of induction, cells were harvested by centrifugation at 3000-rpm for 10-min and re-suspended in 200-mL of lysis buffer {100-mM potassium phosphate (Fisher Scientific), 500-mM NaCl (Fisher Scientific), 10-mM MnCl2 (Fisher Scientific), 2.5-mM imidazole 1-mM PMSF (Alexis) and EDTA free protease inhibitor cocktail tablet (Roche, UK) pH 7.8.}.

Purification of rhFUT6 from Pichia Pastoris Cells

Cells in lysis buffer were disrupted using French Press at 40,000-Kpsi to achieve complete lysis and cell debris was removed by centrifugation step (15000-rpm, 30-min, 4° C.). The supernatant was incubated with 2-mL of Ni-NTA agarose resin (Thermo Scientific) pre-equilibrated with binding buffer {20-mM Tris-HCl (pH 7.8), 500-mM NaCl, 10%-glycerol, 2-mM MnCl2 and 2.5-mM imidazole} for 2-h at 4° C. (batch procedure). After incubation, the resin was collected by low speed centrifugation and loaded onto a pre-equilibrated Polypropylene Column (Qiagen). The column containing rhFTVI bound to resin was then washed with 50-mL washing buffer {20-mM Tris-HCl (pH 7.8), 500-mM NaCl, 10% glycerol, 2-mM MnCl2 and 5-mM imidazole}. The bound protein was eluted by 15-mL of elution buffer {20-mM Tris-HCl (pH-7.8), 150-mM NaCl, 10% glycerol, 2-mM MnCl2 and 400-mM imidazole}. The elution fraction was then concentrated to 0.25-mL by using Amicon concentrator (10-kDa) (PALL). All fractions including the crude extract, the flow through, the washing and the elution fractions were checked for rhFTVI expression using 4-20% SDS-PAGE (Criterion Bio-Rad). rhFTVI was detected in the elution fraction only.

### Determination of Protein Concentration

The protein concentration was determined by using bovine serum albumin as standard. Samples of defined albumin concentrations were prepared, and then the same volume was run in SDS-PAGE gel with recombinant FUT6. After that the intensity profile of the bands were measured by Image J software and blotted the curve (intensities with concentration). FUT6 concentration was calculated by using the equation from the curve.

### Sodium Dodecyl Sulfate Polyacrylamide Gel Electrophoresis SDS-PAGE

The proteins were separated on 4-20% SDS PAGE Criterion Tris Glycin Precast Protein Gels (Biorad). The samples (crude extract, washing fraction and elution fraction) mixed with 1× NuPAGE LDS Sample Buffer (Invitrogen) and 5% betamercaptoethanol as reducing agent then heated 10 min at 75° C. After that it loaded in the gel using 20 uL and for 45 minutes at 120V. Gels were stained with SimplyBlue Safe Stain (Invitrogen) for one hour and distained with water for another one hour.

### Immunoblotting

A polyacrylamide gel was run using elution fraction, transferred by electroborted on PVDF membrane at 0.39 A for 1:20 h. The resulting membrane was blocked with Tris Buffer Saline Tween-20 (20 mM Tris, 137 mM NaCl, 0.1% Tween-20, PH 7.6) containing 5% non-fat milk 1-2 h at room temperature and then incubated with anti-FUT6 antibody (1:1000; Abcam) 1 h at room temperature, followed by incubation in secondary goat anti-rabbit-horseradish peroxidase (1:20000) antibody. Detection was performed using standard chemiluminescence method by incubate the membranes with chemiluminescence reagent for 5 minutes. The same procedure was done with K562 lysate treated with FUT6 that then incubated with chimera E selectin-Ig.

### Mass Spectrometry for Purified FUT6 Protein

Mass spectrometry Sample Preparation (MS): Briefly, eluted fraction was separated using 4-20% SDS-PAGE gels and the protein bands stained visualized by Commassie Stain. After that, all bands were cut that were in range of 70 kDa, 50 kDa, 37 kDa, 25 kDa and 15 kDa. The fractionated bands distained using distaining solution, the gel incubated with trypsin overnight at 37° C. The resulting peptides extracted using extraction buffer that contains 5% acetonitrile, 95% water, 0.1% formic acid. The peptides dried using speed vacuum until approximately lul sample volume. The peptides fractionated by nano-flow LC and analyzed using a LTQ Orbitrap Mass Spectrometer.

### Deglycosylation Assay

FUT6 was treated with 20 mU/ml Peptide-N-Glycosidase F (PNGase-F, Biolabs). The reaction started by denaturation with 1× Glycoprotein Denaturing Buffer (0.5% SDS, 40 mM DTT) at 100° C. for 10 minutes. After the addition of NP-40 and GlycoBuffer 2, twofold dilutions of PNGase F were added and the reaction mix was incubated for 1 hour at 37° C. As a control, each treatment was performed under the same conditions with no added enzymes. Separation of reaction products were visualized by SDS-PAGE with comparing with untreated FUT6.

### Fucosyltransferase Activity Assay

The specific activity of the purified FTVI enzyme was determined by using the Glycosyltransferase Activity Kit (Promega), as per the manufacturer's instructions. Briefly, a serial dilution starting at 5-μL of recombinant FTVI enzyme was prepared in six wells. To the wells the following was added: 250-μM GDP-Fucose (Sigma), 125-μM of N-acetyl-D-lactosamine (Sigma) were mixed in 25-μL reaction buffer (25-mM HEPES (pH 7.5), 5-mM MnCl2 and HBSS) and one reaction well with no FTVI used as a negative control. The reactions were then incubated at room temperature for 1 h. Following this incubation, 25-uL of GDP detection reagent was added to each well in order to convert GDP that was generated from the reaction to ATP which is then measured using luciferase/luciferin reaction. The light generated can then be determined using a luminometer. Luminescence correlates to GDP concentrations, which is determined using a GDP standard curve. All measurements were performed in triplicate

FUT6 exofucosylation

For treatment of cells (K562, MSCs, iPS-HSPCs) with rhFTVI enzyme, cells were harvested, washed 2× with Hank's Balanced Salt Solution (HBSS), and resuspended at a density of 1×10⁶ cells/mL in FTVI reaction buffer {25-mM HEPES (pH 7.5) (Gibco Invitrogen), 0.1% human serum albumin (Sigma-Aldrich), 0.5-mM GDP-fucose (Sigma), 5-mM MnCl2} and appropriate amount of purified rhFTVI enzyme in HBSS. Cells were incubated at 37° C. for 30-min. Buffer only controls excluding the rhFTVI enzyme were used as a negative control. After the reaction, the cells were washed 2× with HBSS and 10-mM EDTA and used immediately for downstream experiments.

### Flow Cytometry

Treated and un-treated (negative control) K562 cells were added in 96 wells plate and stained with Cutaneous Lymphocyte Antigen (PE-HECA-452) antibody to estimate the expression of sLe^{x} structure on the surface of K562 at a concentration of 1 μg/mL for 30 minutes at 4° C. After the incubation, the cells were harvested and suspended in FACS buffer continued 10 mM EDTA, 5% FBS and HBSS to wash them twice with 200 uL/well then analyzed for surface-marker expression using FACSCanto II platform and FlowJo software.

### Immunoprecipitation of E-Selectin Ligands

The cell lysates from FUT6-K562 cells and untreated cells were precleared by incubating it with 30 μL Dynabeads Protein G for 2 hours at 4 C with constant rotation to remove any non-specific binding between the Dynabeads and the lysates. Then the lysates immunopercipated with incubated CD44mAb, CD43mAb and PSGL-1mAb separately with Protein G overnight at 4 C. The supernatant was collected to verify the efficiency of IP while the complex lysate-antibody-beads washed three times with lysis buffer. Then the complex resuspended in 2×LDS and 10% β-mecaptoethanol, followed by 10 minutes heating at 75 C. The samples then were applied to western blot analysis by using E-Ig. (Refer to Western blot protocol above)

### Stamper-Woodruff Assay

E-selectin was spotted on glass slides for 4 hours at 4° C. and fixed with 3% glutaraldehyde followed by 0.2M lysine blocking then the slides were incubated in RPMI 1640, 5 mM CaCl2 and 2% FBS until the analysis. Treated and un-treated K562 with FUT6 were washed with HBSS, the cells were cytospun on slides that coated with E-selectin for 30 minutes at 4° C. and allowed to interact for 30 mins at 4° C. with mild rotation (80 rpm). To exclude the possibility that the treated K562 are interacting nonspecifically with E-selectin, EDTA reaction was used as a negative control binding.

### Results

### Determining the Transformation Efficiency of FUT6-pPinka-HC Vector

Pichia Pastoris cells were plated on two different agars, one containing Yeast Extract Peptone Dextrose (YPD) and the other containing Dextrose-YNB medium without Adenine. Using the FUT6-pPinka-HC vector constructed as described above, stable transformations were made. White and slightly pink colonies were observed on a selection plates upon transformation (data not shown). The pink colonies expressed low levels of the ADE2 gene product, while the white colonies expressed higher amounts of the ADE2 gene products (data not shown).

White colonies were selected in order to more reliably ensure that the gene was integrated. It should be noted that the ADE2 gene on the plasmid enabled the Pichia Pastoris to grow on minimal medium lacking adenine whereas the parent Pichia Pastoris cells would not grow on this minimal medium (data not shown). Following selection of these colonies, the colonies were analyzed for the integration of the plasmid by performing PCR using κ′ and 3′AOX1 primers corresponding to the flanking sequences of the native promotor AOX1 gene as discussed herein.

### Expression and Purification of FUT6

The human FTVI gene is located on chromosome 19p13.3 and has six exons (Cameron et al., 1995). The human FTVI gene encodes for a 359-amino acid (aa) protein that includes an N-terminal region composed of a cytoplasmic tail and a signal-anchor for type II membrane sequence while the C-terminal region consists of a luminal sequence containing a catalytic domain (325 aa) and a stem domain that is adjacent to a membrane-spanning region. A number of truncation studies of fucosyltransferases demonstrated that the N-terminal region is not required for activity, and thereby can be deleted without adverse effects on enzyme activity while changes in the C-terminal catalytic domain results in inactivity (Johnson et al., 1995). An expression construct was designed that spans the catalytic domain of human FTVI (35-359 aa) with a Pichia pastoris secretion signal, the α-mating factor, to induce effective secretion. In addition, the vector includes a 6×Histidine (His)-tag added to the N-terminus of rhFTVI to help in its purification. rhFTVI was produced by Pichia pastoris according to standard protocols (Materials and Methods). Briefly, electroporation was used to transformed cells prior to metabolic phenotype selection. White colonies indicating higher expression of rhFTVI gene, were picked and the presence of functional expression units was confirmed by small-scale cultures induced with 0.5% methanol. Lysates of the Pichia pastoris cells were then prepared and tested for their expression of rhFTVI enzyme.

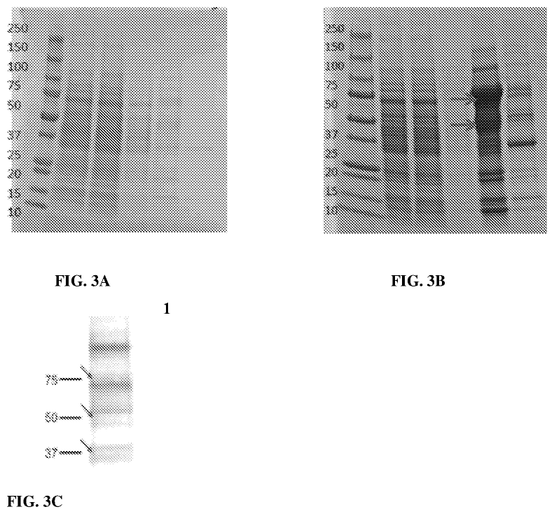

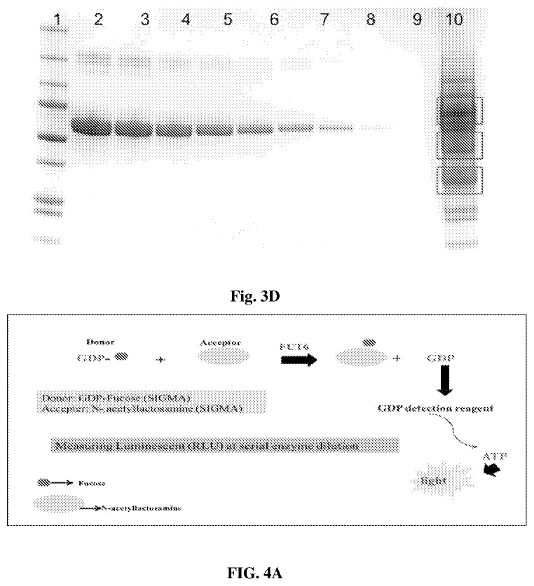

The expression levels of functional FUT6 enzyme in 0.5% methanol-induced recombinant Pichia pastoris cells were found to be highest on the seventh day following induction in BMMY media (data not shown). Cell lysates were then applied to 2 mL Ni-NTA agarose resin affinity column to trap histidine (His tag on FUT6). The columns were washed and the captured enzyme was then eluted using 250 mM imidazole and 400 mM imidazole consecutively. Prior to dialysis, the fractions were concentrated 30 fold. The eluted concentrated fractions were then run along with the crude extract, flow through and washing fractions on an SDS-PAGE gel as illustrated in FIG. 3A. In addition a fraction from the media was also run in order to determine whether the enzyme was secreted or retained with-in the cells (data not shown).

Following the concentration of the eluate, the FUT6 enzyme appeared to be considerably purified following the elution using 250 mM imidazole (indicated by the arrows in FIG. 3B). However, the sample contained some impurities and was not detected in the culture supernatant. The FUT6 enzyme was localized within the Pichia pastoris cells and was not secreted into the media (data not shown). Interestingly, despite inclusion of the Pichia pastoris secretion signal, the α-mating factor in the construct used to transfect Pichia pastoris cells (to induce effective secretion) the rhFTVI enzyme was localized within the Pichia pastoris cells and was not secreted into the media.

### Recombinant FUT6 Enzyme Characteristics

In order to detect and identify recombinant FUT6 protein and to determine its molecular weight, two methods were utilized, one immunoblotting with anti-Fut6 and other was mass spectrometry (MS).

### Determination of Recombinant FUT6 Molecular Weight

Molecular weight of FUT6 expressed by Pichia Pastoris, was determined using a Western blot analysis. Detection using anti-FUT6 antibody revealed a pattern of two major bands with molecular weights corresponding to 47 kDa and 43 kDa and two minor bands at 40 kDa and 37.5 kDa (FIG. 3C), both representing putative degradation products appeared as about 48 kDa protein and 37 kDa.

As stated above, the rhFTVI was expressed intracellularly and not secreted in the media and thereby the expected molecular weight is −50-kDa. Interestingly, the N-tagged rhFTVI at −70-kDa was higher than the predicted size likely due to differential posttranslational modifications of four potential N-linked glycosylation sites of the rhFTVI protein. To decipher which bands represent FTVI, a mass spectrometry (MS) approach was used to confirm the purification of the rhFTVI enzyme as well as confirm the results obtained from the Western blot analysis.

### FUT6 Identification by Mass Spectrometry

Purified FUT6 was run on an SDS-PAGE and the bands were prepared (data not shown) as described herein. The raw data was converted to Mascot Generic Format files and a search using the online Mascot database was performed. The MS analysis suggested that the FUT6 protein was found corresponding to molecular weights 75 kDa, 48 kDa and 37 kDa with 52%, 56% and 32% coverage respectively. FUT6 was not detectable at 25 kDa and 15 kDa bands. According to the Western blot in FIG. 3C these molecular weights indicate that the bands just below 50 kDa and at 37 kDa are likely FUT6 protein.

### Determination of FUT6 Protein Concentration

Recombinant FUT6 protein concentration was calculated using bovine serum albumin standards. Since the FUT6 appears to correspond to different molecular weights, this required a reliable method for concentration estimation. SDS-PAGE was performed (FIG. 3D) and the intensity profile was blotted against the defined concentration of albumin Recombinant FUT6 concentration was calculated using linear regression equation from BSA standard titration Y=262424x+3473.9. 75 kDa band concentrations were −1.97 mg/mL, 50 kDa band was −1.1 mg/mL and the 37 kDa band was −0.8 mg/mL. Total FUT6 concentration was found to be −4 mg/mL.

### Determination of Fucosyltransferase 6 Activity

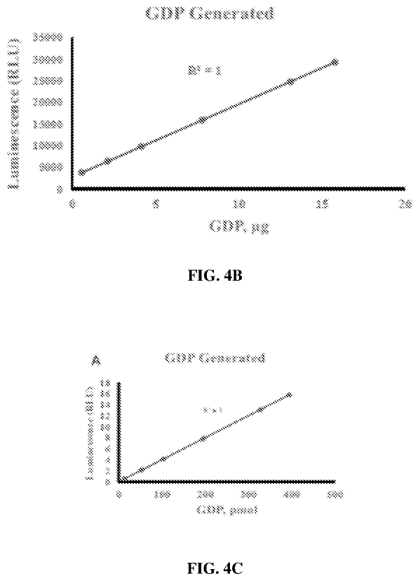

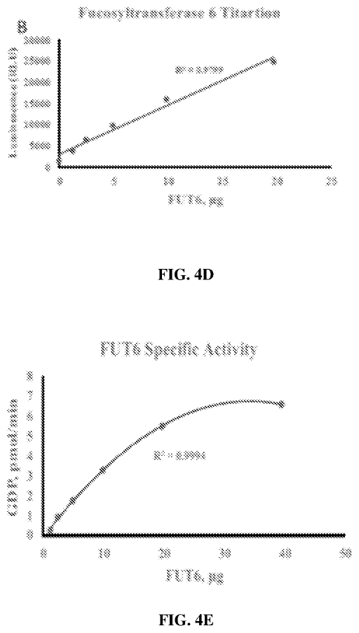

The enzyme assay for FUT6 was conducted as described above. The enzymatic activity was assessed using a luciferase based assay. This assay relies on measuring GDP released from the glycosyltransferase reaction. One unit (U) of enzyme activity corresponds to the transfer of 1 pmol of sugar (GDP-fucose) from the donor to the acceptor per min at 37° C. (refer to FIG. 4A). To determine the specific activity of FUT6 enzyme, a GDP standard curve was prepared with concentration range (0-25 μM) in a total volume of 25 uL per reaction (FIG. 4B). The GDP solutions were made from 10 mM GDP stock solution (provided with the assay kit) using buffer containing 25 mM HEPES, 5 mM MnCl2, pH 7.5 and HBSS. To 25 μl of a GDP standard solution, 25 μl of the GDP detection reagent was added and the corresponding luminescence was measured (Table 2).

TABLE 2
GDP titration using GDP-Glo Assay.

| | GDP μM | | | | | | | | | | |
|---|---|---|---|---|---|---|---|---|---|---|---|
| | 25 | 12.5 | 6.3 | 3.1 | 1.56 | 0.78 | 0.39 | 0.2 | 0.05 | 0.02 | 0 |
| RLU | 3918 | 3115 | 1871 | 1151 | 7272 | 4276 | 2030 | 1174 | 680 | 624 | 490 |

RLU; Relative Luminescence Unit.

A linear relationship was observed between the luminescent signal and the amount of GDP in the reaction buffer up to 25 μM GDP. In order to determine the activity of FUT6 in U/mL, the amount of GDP generated of the reaction was determined using a linear regression equation from GDP standard titration Y=1673.7x+2859.2. Recombinant FUT6 enzyme was titrated in 25 μl in GT reaction buffer (25 mM HEPES, 5 mM MnCl2, pH 7.5 and HBSS) in a 96-well plate in the presence 40 μM Ultra-Pure GDP-Fucose.

After a 1 hour incubation at 23° C., GDP-Glo GT Assay was performed using 25 μl of GDP detection reagent at room temperature as described in materials and methods. Luminescence was recorded using a GloMax 96 Microplate Luminometer (FIGS. 4C and 4D). As shown in FIG. 4B, a linear relationship was observed between the luminescent signal and the amount of FUT6. Specific activity of the FUT6 enzyme was calculated using the curve in FIG. 4E as pmol of GDP produced/min/ug of enzyme. The overall activity of FUT6 was ˜13000 U/mL (one unit (U) of enzyme activity corresponds to the transfer of 1 pmol of sugar (GDP-fucose) from the donor to the acceptor per min at 37° C.).

### FUT6 Exofucosylation

A number of different acute myeloid leukemic cell lines were first tested for their native sLe^{x} expression and E-selectin binding [E-selectin-hIg chimera; E-Ig] by flow cytometry. All cell lines (HL-60, THP1 and KG1a) with the exception of K562 cells expressed sLe^{x} as indicated by the reactivity of monoclonal antibodies (mAbs), HECA-452 and CD15s. The expression of sLe^{x} correlated with the ability of cells to bind E-Ig (data not shown). K562 cells were therefore chosen as a model cell line to determine whether the activity of GTs leads to the creation of sLe^{x} structures on cells. Biosynthesis of sLe^{x} involves (i) a2,3-sialyltransferases (encoded by ST3GAL genes ST3GALIII and ST3GALIV) and (ii) a1,3-fucosyltransferases (encoded by FT genes FTIII, FTIV, FTV, FTVI, FTVII) (Ma et al., 2006). Moreover, to better understand the expression of the GTs, FTs and ST3GALs, necessary for the terminal monosaccharide additions of fucose and sialic acid in the biosynthesis of sLe^{x}, an mRNA expression analysis of these genes in K562 cells was performed by real-time semi-quantitative PCR.

The data revealed that ST3GALIII and ST3GALIV transcripts were relatively highly expressed in K562 cells. The expression of the α1,3-FTs, i.e. FTIII, FTIV, FTV, FTVI and FTVII (de Vries et at, 2001), were found to be expressed at very low levels or absent in K562 cells compared to the α1,2-FTs, FTI and FTII, which are responsible for H blood group antigen (Rouquier et al., 1995). Interestingly, the α1,6-FT, FTVIII, was also expressed in these cells. FTIV and FTVII are the main human FTs expressed in leukocytes responsible for the creation of functional selectin ligands (Wagers et al., 1997) but both of these enzymes were found to be expressed at low levels in the K562 cells.

K562 cells were treated with FUT6 in a reaction buffer that contained GDP-fucose as donor for fucose, and MnCl2 as cofactor for the enzyme. Following treatment of the cells with the FUT6 enzyme, the cells were stained using antibodies (HECA452 clone) that recognize the sLe^{x} carbohydrate structure and analyzed by flow cytometry. FIG. 3.12 shows that following treatment, the HECA452 antibody recognizes K562 cells where prior to treatment with the FUT6 enzyme, they were not.

K562 cells express low amount of sLe^{x} prior to treatment and ex-vivo fucosylation was sufficient to decorate K562 cells with sLe^{x} structures.

Optimization of Ex-Vivo Fucosylation Treatment of K562 Cells

Mn²⁺ was used in the enzymatic reactions as catalyst for high efficiency fucosyltransferase activity, but Mn²⁺ could induce prominent cell death. In order to minimize cell death, the most effective concentration was determined by titrating the concentrations in the range (0-5 mM) of MnCl2. The enzyme is most active when either no glycerol is used as a stabilizer or with 50% glycerol as a stabilizer. In addition, in the absence of glycerol, the enzyme was sufficiently active at MnCl2 concentrations corresponding to 3-4 mM.

Optimal conditions for treatment that maintained a high percentage of cell viability without affecting activity was to store the enzyme lyophilized without glycerol and use a MnCl2 concentration from 3-4 mM.

### Assessment of Glycoprotein Ligands Created by FUT6 Treatment

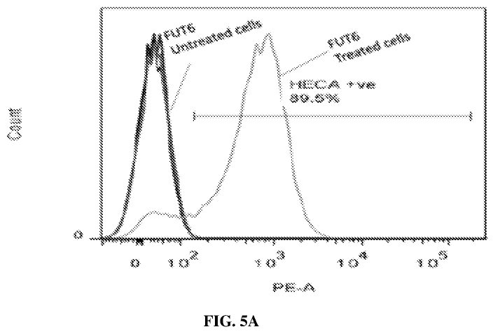

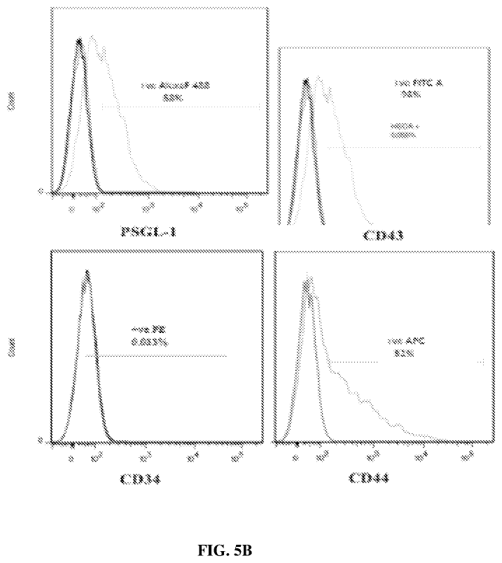

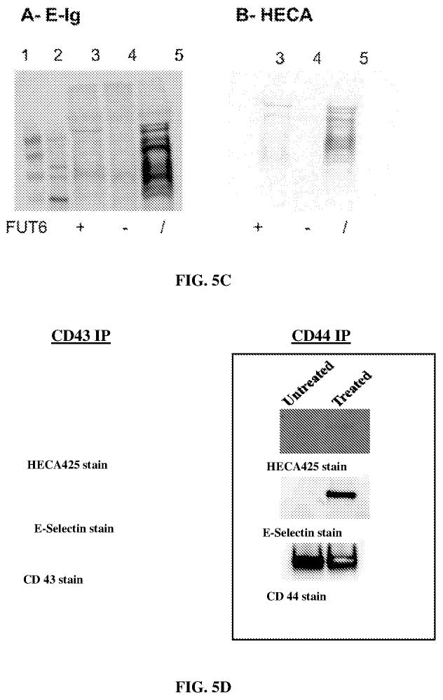

Subsequent studies sought to characterize which potential E-selectin protein ligands were expressed on K562 cells by staining cells with antibodies directed against known glycoprotein ligands, namely, CD44, CD43 and PSGL-1 (Merzaban et al., 2011), and analyzed their expression by flow cytometry. To determine the potential E-selectin ligands created by FUT6 treatment, K562 cells were stained with antibodies directed against known ligands (19) (namely CD44, CD43, and PSGL-1) and analyzed their expression using flow cytometry (FIG. 5B). As shown in FIG. 5B, K562 cells express PSGL-1, CD43 and CD44.

It should be noted that expression of these glycoproteins did not equate to expression of sLe^{x} or to functional E-selectin binding. To this end, K562 cells were incubated with the purified rhFTVI enzymes. Following treatment with either of the rhFTVI enzymes, the cells were stained using antibodies that recognize the sLe^{x} (HECA452) and CD15s (CSLEX1) as well as with E-Ig, and analyzed by flow cytometry. Following treatment, sLe^{x} expression was increased as denoted by the expression of HECA-452 and CSLEX1. Furthermore, rhFTVI treatment allowed the cells to bind to E-Ig. The specificity of binding with E-Ig was demonstrated when the interaction was abolished using EDTA to chelate Ca²⁺, an essential cation for mediating binding of E-selectin to its ligands (AbuSamra et al., 2017)

### Western Blot Analysis of E-Selectin Ligands Created Following FUT6 Treatment of K562 Cells

To determine the glycoproteins that act as E-selectin ligands following FUT6 treatment of K562 cells, a western blot was performed and the blot was probed with a recombinant E-selectin-Ig chimera (E-Ig) to measure its ability to bind proteins from treated cell lysates. Lysates from FUT6 treated K562 was prepared and blotted onto two separate membranes and stained with E-selectin-Ig chimera (E-Ig) or HECA452 antibody that recognizes sLe^{x} (FIG. 5C). Kgl a cell lysates were used as positive controls for these experiments as these cells carry high levels of functional E-selectin ligands (27).

FUT6 treatment was sufficient to induce sLe^{x} structures on proteins and the formation of E selectin glycoprotein ligands that appear at different molecular weights 120 kDa for CD43, 120-240 kDa for PSGL-1 and 100 kDa for CD44. This appears to indicate that these E-selectin ligands were created following treatment.
To analyze and more directly identify of E-selectin glycoproteins created by FUT6 treatment, equal amounts of each ligand were immune-purified before and after treatment and assessed E-Ig binding activity by Western blot (FIG. 5D).

Both CD44 and CD43 glycoproteins were decorated with a1,3 fucose after using α-1,3 linkage specific FUT6 treatment (FIG. 5D). These data indicated that after fucosylation of K562 cells, E-selectin ligands were created as indicated by strong sLe^{x} expression and E-Ig binding. CD44 and CD43 may lack E-selectin binding in untreated K562 cells due to the absence of α-1,3-fucose at the terminal sialylated lactosamine unit.

Overall, these studies show that the rhFUT6 produced from yeast were able to add fucose to ligands in order to create sLe^{x} and allow for E-selectin to bind cells that previously did not bind.

Functional E-Selectin Ligands were Created on K562 Cells Following Treatment with FUT6

To establish if the ligands created following FUT6 treatment on K562 cells were functional in flow based assays, the Stamper Woodruff assay was performed. Slides were coated with E-Ig and then both untreated K562 and treated FUT6-K562 cells were added onto slides and allowed to rotate for 30 min at 80 rpm. Following the incubated time, the numbers of rolling cells/mm² in seven distinct fields of view were counted (data not shown, FIG. 5D), K562 cells treated with FUT6 bound E-Ig to a much greater degree than untreated cells or treated cells where EDTA was used to chelate the Ca²⁺ and show specificity, a requirement for mediating binding of selectins to their ligands.

The results illustrate that K562 cells can roll on E-selectin in presence of Ca+² after FUT6 treatment and this behavior was abrogated in the presence of 20 Mm EDTA.

Mesenchymal Stromal Cells and Hematopoietic Stem/Progenitor Cells Derived from Induced Pluripotent Stem Cells Gain Selectin Ligands Following Ex Vivo Treatment with rhFTVI

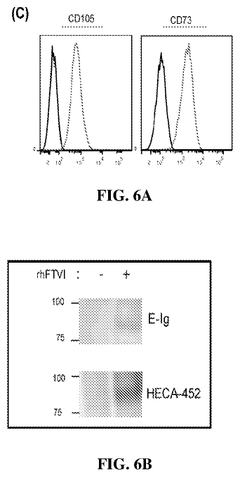

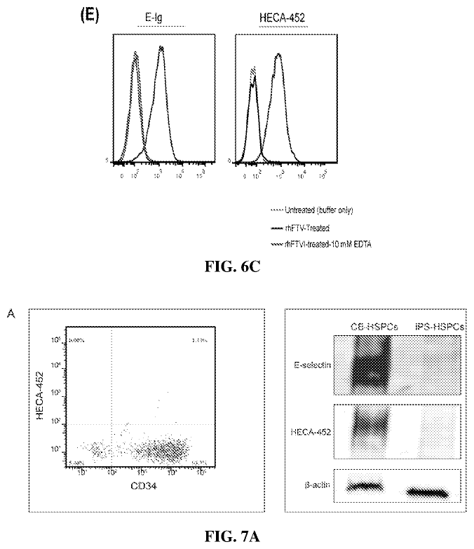

Often adult stem cell populations are inadequately a 1,3-fucosylated and so exhibit homing defects (Hidalgo and Frenette, 2005; Sackstein et al., 2008; Robinson et al., 2012; Merzaban et al., 2015; Popat et al., 2015; Chou et al., 2017). To test the ability of rhFTVI to create sLe^{x} structures on primary mesenchymal stromal cells (MSCs), human MSCs were treated with the fucosyltransferase enzyme and sLe^{x} formation and E-selectin binding measured by flow cytometry. As shown in FIG. 6A, human MSCs displayed characteristic surface markers, CD105 and CD73. Following treatment with rhFTVI, the MSCs were lysed and assessed for E-Ig binding and HECA-452 reactivity by Western blot. The major band observed was at −80-kDa following treatment (FIG. 6B), which is in agreement with previous studies indicating that the major glycoprotein E-selectin ligand on these cells is the standard glycoform of CD44, hematopoietic cell E- and/or L-selectin ligand (HCELL) (Sackstein et al., 2008; Lopez-Lucas et al., 2018). Flow cytometric analysis also confirmed the gain in expression of sLe^{x} structures as well as E-selectin binding following treatment with rhFTVI (FIG. 6C).

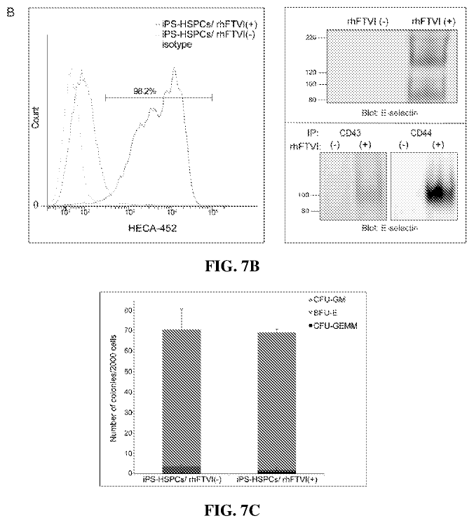

Hematopoietic stem cell transplantation (HSCT) is offered to patients with life-threatening blood disorders and hematological malignancies. In vitro studies have shown that iPS-derived HSPCs (iPS-HSPCs) behave much like somatic HSPCs exhibiting robust clonal proliferation and multilineage hematopoietic capacity. Thus, iPS hold great promise since they are amenable to large-scale production and can overcome the challenge of finding immune-compatible donors. Nonetheless, the utilization of iPS-HSPCs in HSCT is limited by the relative scarcity of finding them in the bone marrow following transplantation. Studies were conducted to determine whether the low engraftment of iPS-HSPCs could be due, at least partially, to a deficiency in migration and thus in the expression of properly glycosylated selectin ligands. As illustrated in FIG. 7A (left panel), iPS-HSPCs lack the sLe^{x} epitope as indicated by the absence of HECA-452 staining. Moreover, the absence of sLe^{x/a} was consistent with the absence of E-selectin binding activity as shown by Western-blot analysis (n=3) (FIG. 7A, right panel). Inadequate fucosylation of selectin ligands can result in poor HSPCs homing and engraftment. However, this defect can be circumvented by exogenous fucosylation of the ligands. To test whether the iPS-HSPCs express adequate amounts of α(1,3)-fucosyltransferase compared to cord blood HSPCs, q-PCR analysis was performed to quantify the relative expression of FT-VII, which is widely expressed on hematopoietic cells including CD34+ cells from BM and appears to be the dominant FT responsible for producing leukocyte selectin ligand activity. The data demonstrated that iPS-HSPCs express a significantly lower amount of FT-VII (49.9±4.7 fold; data are mean±SD, n=3, p-value=0.007) than HSPCs from cord blood. Given this stark difference in fucosyltransferase expression, studies were conducted to determine if ex vivo treatment of the iPS-HSPCs with rhFT-VI would be sufficient to create E-selectin ligands. Following treatment with rhFTVI, iPS-HSPCs gained HECA-452 reactivity indicating sLe^{x/a} structures were created (FIG. 7B, left panel). The ability of the fucosylated ligands to bind E-selectin was confirmed by Western-blot analysis (FIG. 7B, upper right panel). Furthermore, immunoprecipitation of the known E-selectin ligands on HSPCs shows that both CD43 and CD44 were decorated with HECA-452 antigenic determinants following treatment (FIG. 7B, lower right panel). To determine whether the treatment affected the clonogenic activity of iPS-HSPCs, clonogenicity assays were performed. As evident in FIG. 7C, the treatment did not significantly influence changes in the clonogenic ability of the iPS-HSPCs colonies compared to control treated cells (n=3, p-value=0.91).

### Discussion

Ex vivo glycan engineering of glycoprotein ligands on stem/progenitor cells creates glycan structures that help guide infused cells to endothelial beds that express E-selectin, thereby enabling efficient vascular delivery of these cells to sites where they are needed. Creating efficient active GTs is not a trivial task. Eukaryotic expression systems are preferred over bacterial systems in the production of GTs. This work outlines a novel method that is used to express and purify recombinant human FTVI using the Pichia Pastoris yeast expression system that overcomes several disadvantages of existing systems, related to the ease of expression and cost. Typically such purification procedures require synthetic columns with multiple steps for the purification of proteins and are associated with high costs for materials especially for expression in mammalian systems (i.e. CHO cells).

To this end, following the expression of rhFTVI in Pichia pastoris, the samples (supernatant and cell lysates) were concentrated and purified using a single step nickel column; the samples were then dialyzed and the rhFTVI was characterized. FTVI was detected intracellularly. When considering conditions for protein stabilization, it is best to closely examine the in vivo environment of the protein to be handled. In addition, protein solutions are more stable when maintained at higher concentrations, preferably >1 mg/mL, since the native structure in more preserved at these concentrations.

The concentrated purified rhFTVI enzyme produced from Pichia pastoris yeast was detected at various molecular weights following separation of eluted proteins on an SDS-PAGE gel. Based on the amino acid sequence, the expected molecular weight of the secreted Pichia pastoris form of the enzyme was ˜38-kDa while the non-secreted intracellular form, likely represented as a homodimer (Borsig et al., 1998), was ˜76-kDa. Mass spectrometry analysis confirmed that the rhFTVI enzyme from the Pichia pastoris yeast system were specifically found at ˜75-kDa, ˜50-kDa and ˜38-kDa.

Using K562 cells as a model, the data showed that the recombinantly expressed FUT6 created sLe^{x} structures that bind E-selectin. Indeed, an analysis of GTs in K562 cells revealed that although sialyltransferases that support the synthesis of sLe^{x}, ST3Gal-III and —IV, were expressed, low to no expression of fucosyltransferases that support sLe^{x} synthesis were found. Interestingly, the main E-selectin ligand that formed following rhFTVI treatment of K562 cells was CD43 although small amounts of CD44 and PSGL-1 also served as E-selectin ligands.

The data in this application show that following treatment of K562 cells or MSCs with rhFTVI from \Pichia pastoris resulted in cells expressing sLe^{x} positive epitopes that bound E-selectin. iPS-HSPCs lack the appropriate antigenic determinant for HECA-452 binding, thus leading to defective binding to E-selectin, a key adhesion molecule important for directing the migration of stem cells to the bone marrow which in agreement with. The data shows that this lack of HECA-452 is due to inadequate expression of the appropriate FTs, specifically FT-VII. Fucosylation using rhFTVI was sufficient to decorate iPS-HSPCs with HECA-452 antigenic determinants leading to substantial increases in E-selectin binding activity. This ex vivo approach is likely more effective than using the FT mRNA-mediated glycoengineering protocol since the stem cells' phenotypic characteristics and viability are better preserved. Recently, reports have shown that ectopic expression of master hematopoietic transcription factors in iPS cells leads to the derivation of engraftable HSPCs with some potential for migration when injected intrafemorally. Through the development of improved protocols to derive HSPCs and such technologies as those outlined here, these sources of HSPCs could be that much closer to clinical use.

The present studies outline a simple approach for purifying high quality and quantity active GT enzymes from the yeast expression system using only single step purification IMAC (immobilized metal affinity chromatography).

1. A method for recombinantly producing an enzymatically active glycosyltransferase enzyme comprising introducing into Pichia pastoris, a vector comprising a gene encoding the catalytic domain of the GT enzyme, operably linked to one or more expression control and a Pichia pastoris secretion signal and a polyhistidine tag to produce a recombinant Pichia pastoris and culturing the recombinant Pichia pastoris for an effective time to express the GT enzyme.

2. The method of claim 1, wherein the Pichia pastoris strain with comprises an ade2 deletion.

3. The method of claim 1, wherein the Pichia pastoris secretion signal comprises the α-mating factor.

4. The method of claim 1, wherein the polyhistidine tag comprises a 6×Histidine (His)-tag added to the N-terminus of the gene encoding the GT enzyme.

5. The method of claim 1, wherein the GT transferase enzyme is a human alpha-(1,3)-fucosyltransferase

6. The method of claim 5, wherein the GT enzyme is human alpha-(1,3)-fucosyltransferase 6 (FUT6).

7. The method of claim 6, wherein the GT gene in the vector encodes the amino acids 35-359 of human alpha-(1,3)-fucosyltransferase 6 (FUT6)

8. The method of claim 1, further comprising purifying the recombinantly expressed GT enzyme from the host cells.

9. The method of claim 8, wherein the purification step does not comprise use of a GDP-hexanolamine column or wherein the enzyme is purified using immobilized metal affinity chromatography (IMAC).

10. (canceled)

11. The method of claim 9, further comprising lyophilizing the purified enzyme.

12. A composition comprising recombinantly produced GT enzyme in a buffer, wherein the buffer optionally comprises 0% glycerol or 50% glycerol.

13. The composition of claim 12, wherein: (a) the buffer comprises 0% 50% glycerol or (b) the GT enzyme is human FUT6.

14. (canceled)

15. The composition of claim 13, comprising amino acids 35-359 of FUT6.

16. The composition of claim 12 further comprising manganese in a concentrations between 3 and 4 Mm.

17. The composition of claim 15, wherein the manganese is added in the form of MnCl2.

18. A method for improving migration of cells in need thereof, comprising contacting the, with the composition of claim 12, the composition further comprising a substrate for the GT enzyme, for an effective amount of time to catalyze transfer of the substrate onto an acceptor site on the cells.

19. The method of claim 18, wherein the enzyme is FUT6 and the substrate is GDP-fucose.

20. The method of claim 18, wherein the cell is selected from the group consisting of hematopoietic stem cells, neural stem cells, induced pluripotent stem cells, skeletal myoblasts, bone marrow cells, circulating blood-derived progenitor cells, endometrial mesenchymal stem cells, adult testis pluripotent stem cells, mesothelial cells, adipose-derived stromal cells, embryonic cells, induced pluripotent stem cells, and bone marrow.

21. The method of claim 20, wherein the cells are induced pluripotent stem cells.

22. The method of claim 20, wherein the cells are mesenchymal stromal cells or hematopoietic stem/progenitor cells.
